(19) United States
(12) Patent Application Publication
(10) Pub. No.: US 2022/0031292 A1
(43) Pub. Date: Feb. 3, 2022

(54) BIOPSY DEVICE WITH INTEGRATED VACUUM RESERVOIR

(71) Applicant: Devicor Medical Products, Inc., Cincinnati, OH (US)

(72) Inventor: Andrew P. Nock, Dayton, OH (US)

(21) Appl. No.: 17/500,185

(22) Filed: Oct. 13, 2021

Related U.S. Application Data
(63) Continuation of application No. PCT/US2020/029512, filed on Apr. 23, 2020.
(60) Provisional application No. 62/837,835, filed on Apr. 24, 2019.

(51) Int. Cl. A61B 10/02; A61B 10/00

(52) U.S. Cl. CPC A61B 10/0283; A61B 10/0275; A61B 10/0096

(57) ABSTRACT

A probe for use with a biopsy device. The biopsy device having a tissue sample holder defining a sample chamber and a holster removably secured to the probe. The probe includes a housing. The housing defining a vacuum chamber within the housing. The vacuum chamber being in communication with the sample chamber of the tissue sample holder.

### PRIORITY

This application claims priority to U.S. Provisional Patent App. 62/837,835 entitled “Biopsy Device with Integrated Vacuum Reservoir,” filed on Apr. 24, 2019, the disclosure of which is incorporated by reference herein.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Other biopsy devices may be fully or at least partially operable without being tethered or otherwise connected with another device.

Merely exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pat. No. 6,626,849, entitled “MRI Compatible Surgical Biopsy Device,” issued Sept. 30, 2003; U.S. Pat. No. 7,442,171, entitled “Remote Thumbwheel for a Surgical Biopsy Device,” issued Oct. 28, 2008; U.S. Pat. No. 8,764,680, entitled “Handheld Biopsy Device with Needle Firing,” issued Jul. 7, 2014; U.S. Pat. No. 9,345,457, entitled “Presentation of Biopsy Sample by Biopsy Device, issued May 24, 2016; U.S. Pub. No. 2006/0074345, entitled “Biopsy Apparatus and Method,” published Apr. 6, 2006, now abandoned; U.S. Pub. No. 2009/0171242, entitled “Clutch and Valving System for Tetherless Biopsy Device,” published Jul. 2, 2009; U.S. Pub. No. 2010/0152610, entitled “Hand Actuated Tetherless Biopsy Device with Pistol Grip,” published Jun. 17, 2010; and U.S. Pub. No. 2012/0310110, entitled “Needle Assembly and Blade Assembly for Biopsy Device,” published Dec. 6, 2012. The disclosure of each of the above-cited U.S. Pat. Nos., U.S. Patent Application Publications, and U.S. Non-Provisional Patent Applications is incorporated by reference herein.

In some circumstances, it may be desirable to use a biopsy device without a tether to a vacuum source, controller, or other peripheral accessory item. For instance, in ultrasonically guided biopsy procedures, a tetherless biopsy device may be desirable due to the nature of the procedure being entirely handheld without the use of support structures, guides, manipulators, or other devices associated with manipulation of the biopsy device. When the biopsy device is entirely handheld, manipulation of the biopsy device might be encumbered by tethers to peripheral items. Thus, a tetherless biopsy device may be desirable in some circumstances.

In circumstances where a biopsy device is tetherless, all components necessary for operating the biopsy device are incorporated into the biopsy device itself in a compact handheld package. This constraint can lead to certain tradeoffs in operation. For instance, for the supply of vacuum, an onboard vacuum pump can be used. One such tradeoff with this configuration is the absence of a vacuum canister that is typically used in tethered biopsy devices. The presence of a vacuum canister adds volume to the vacuum system. This additional volume can provide a smoothing effect to vacuum pressure as more or less vacuum is used by the biopsy device during various operational stages. Without such a vacuum canister in tetherless biopsy devices, the vacuum pressure can be more erratic as the biopsy device moves through various operational stages that are more or less demanding on vacuum. Thus, in the context of tetherless biopsy devices it may be desirable to incorporate features to smooth vacuum pressure as the biopsy device moves through various operational stages.

While several systems and methods have been made and used for obtaining a biopsy sample, it is believed that no one prior to the inventors has made or used the invention described in the appended claims.

### BRIEF DESCRIPTION OF THE FIGURES

While the specification concludes with claims which particularly point out and distinctly claim the biopsy device, it is believed the present biopsy device will be better understood from the following description of certain examples taken in conjunction with the accompanying drawings, in which like reference numerals identify the same elements and in which:

FIG. 1 depicts a perspective view of an exemplary biopsy device;

FIG. 2 depicts a perspective view of the biopsy device of FIG. 1, showing a holster detached from a probe;

FIG. 3 depicts a schematic view of exemplary electrical and/or electromechanical components of the holster of FIG. 2;

FIG. 4 depicts a perspective cutaway view of the probe of FIG. 2;

FIG. 5 depicts an exploded perspective view of the probe of FIG. 2;

FIG. 6 depicts a front cross-sectional view of a needle actuation assembly of the probe of FIG. 2;

FIG. 7 depicts a front cross-sectional view of the probe of FIG. 2; and

FIG. 8 depicts a schematic view showing the relationship between a valve state and a cutter position.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the biopsy device should not be used to limit the scope of the present biopsy device. Other examples, features, aspects, embodiments, and advantages of the biopsy device will become apparent to those skilled in the art from the following description, which is by way of illustration, one of the best modes contemplated for carrying out the biopsy device. As will be realized, the biopsy device is capable of other different and obvious aspects, all without departing from the spirit of the biopsy device. Accordingly, the drawings and descriptions should be regarded as illustrative in nature and not restrictive.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

### I. Overview of Exemplary Biopsy Device

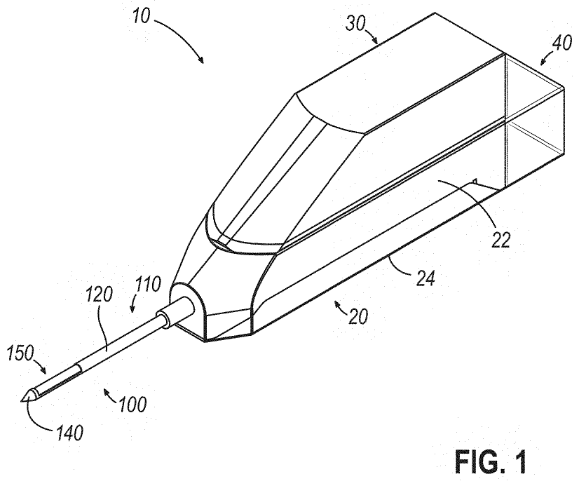

FIG. 1 shows an exemplary biopsy device (10), comprising a probe (20) and a holster (30). It should be understood that biopsy device (10) of the present example is generally configured as a tetherless biopsy device. As such, biopsy device (10) is generally self-contained with all components needed for operation included within either probe (20) or holster (30). Although biopsy device (10) of the present example is shown and described as a tetherless biopsy device (10), it should be understood that the teachings herein can be readily applied to biopsy devices having other configurations including tethered configurations.

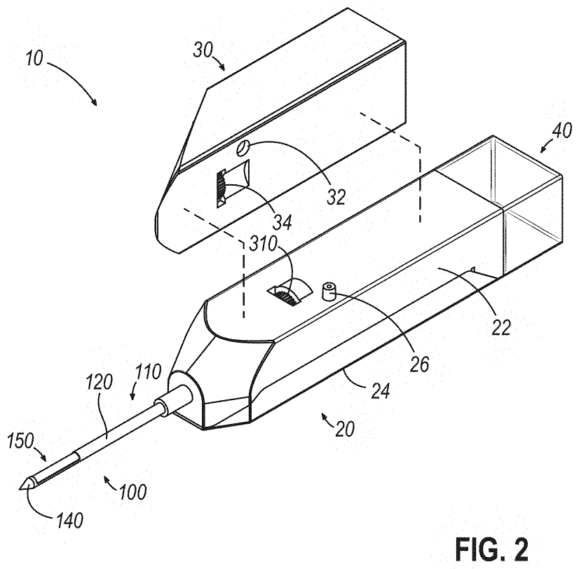

Probe (20) comprises a needle assembly (100) that at least partially extends distally from a casing of probe (20). Needle assembly (100) is insertable into a patient's tissue to obtain tissue samples as will be described below. Biopsy device (10) further comprises a tissue sample holder (40) into which the tissue samples are deposited. By way of example only, probe (20) may be a disposable component and holster (30) may be a reusable component to which probe (20) may be coupled, as is shown in FIG. 2. Use of the term “holster” herein should not be read as requiring any portion of probe (20) to be inserted into any portion of holster (30). Indeed, in one configuration for biopsy device (10), probe (20) may simply be positioned atop holster (30). Alternatively, a portion of probe (20) may be inserted into holster (30) to secure probe (20) to holster (30). In yet another configuration, a portion of holster (30) may be inserted into probe (20). Further still, probe (20) and holster (30) may be integrally formed as a single unit.

In configurations where probe (20) and holster (30) are separable members, a port and/or a seal (32) may be provided on holster (30) to couple with a second port and/or a seal (26) on probe (20) such that the vacuum produced by a vacuum pump (50) within holster (30) may be fluidly connected to probe (20). Holster (30) may also provide a gear (34) or multiple gears which mate to and engage with a corresponding gear (310) on probe (20). It should be understood that the configuration depicted in FIG. 2 that communicates vacuum and motive force between holster (30) and probe (20) is merely exemplary. In some versions, such configurations may be constructed in accordance with at least some of the teachings of U.S. Pat. No. 8,206,316 entitled “Tetherless Biopsy Device with Reusable Portion,” issued Jun. 26, 2012; and/or U.S. Pub. No. 2012/0065542, entitled “Biopsy Device Tissue Sample Holder with Removable Tray,” published Mar. 15, 2012, the disclosures of which are incorporated by reference herein.

With holster (30) and probe (20) connected, vacuum pump (50) can induce a vacuum within needle assembly (100) via tissue sample holder (40) and a tubular cutter (60). However, it should be understood that vacuum may be provided in other ways. For example, vacuum pump (50) may be independent of holster (30) and probe (20) and may simply be coupled by vacuum tubes to appropriate ports on biopsy device (10). Biopsy device (10) may further be configured in accordance with at least some of the teachings of U.S. Pat. No. 8,764,680, entitled “Handheld Biopsy Device with Needle Firing,” issued Jul. 1, 2014; and/or U.S. Pub. No. 2012/0065542, entitled “Biopsy Device Tissue Sample Holder with Removable Tray,” published Mar. 15, 2012, the disclosures of which are incorporated by reference herein. Other suitable structural and functional combinations for probe (20) and holster (30) will be apparent to one of ordinary skill in the art in view of the teachings herein.

### II. Exemplary Holster

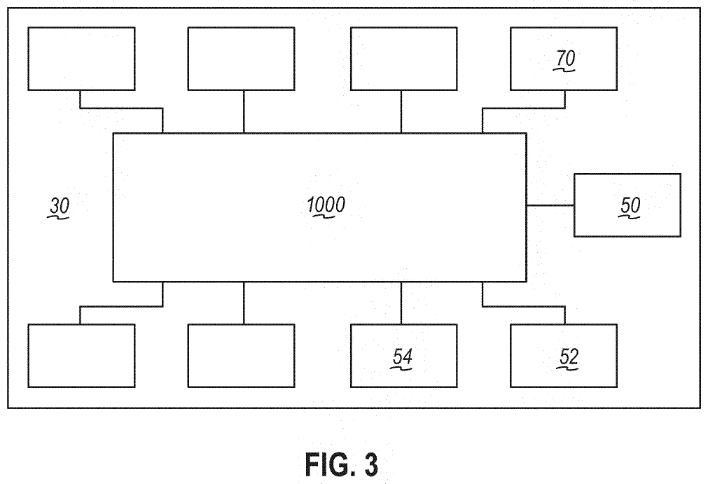

Holster (30), shown schematically in FIG. 3, comprises vacuum pump (50), a motor (70), a control module (1000), a one or more buttons (54), a vacuum sensor (52), and any other suitable electrical and/or electromechanical components. Vacuum pump (50) of the present example comprises a conventional diaphragm pump that is mechanically coupled to motor (70). Vacuum sensor (52) is coupled to vacuum pump (50) or along any vacuum path therefrom such that vacuum sensor (52) can determine the level of vacuum created by vacuum pump (50). Vacuum sensor (52) is electrically coupled to control module (1000) so that vacuum sensor (52) may output signals indicative of the vacuum level to control module (1000). In the configuration shown, motor (70) is operable to translate and/or rotate cutter (60) in response to actuation of one or more of buttons (54), as will be described below, and to activate vacuum pump (50), though this is merely optional and a second motor (not shown) may be provided to run vacuum pump (50). In particular, motor may be coupled to a cutter actuation assembly (300) and may be activated by control module (1000) upon actuating one or more of buttons (54). Such a cutter actuation assembly (300) may rotate gear (34). As noted above, gear (34) meshes with gear (310) in probe (20) thus allowing motor (70) to translate and/or rotate cutter (60). Other various configurations for holster (30) may be provided as will be apparent to one of ordinary skill in the art in view of the teachings herein. By way of example only, the cutter actuation assembly (300) and/or other features of holster (30) and/or probe (20) may be constructed in accordance with at least some of the teachings of U.S. Pat. No. 8,206,316 entitled “Tetherless Biopsy Device with Reusable Portion,” issued Jun. 26, 2012; and/or U.S. Pat. No. 8,764,680, entitled “Handheld Biopsy Device with Needle Firing,” issued Jul. 1, 2014, the disclosures of which are incorporated by reference herein.

### III. Exemplary Probe

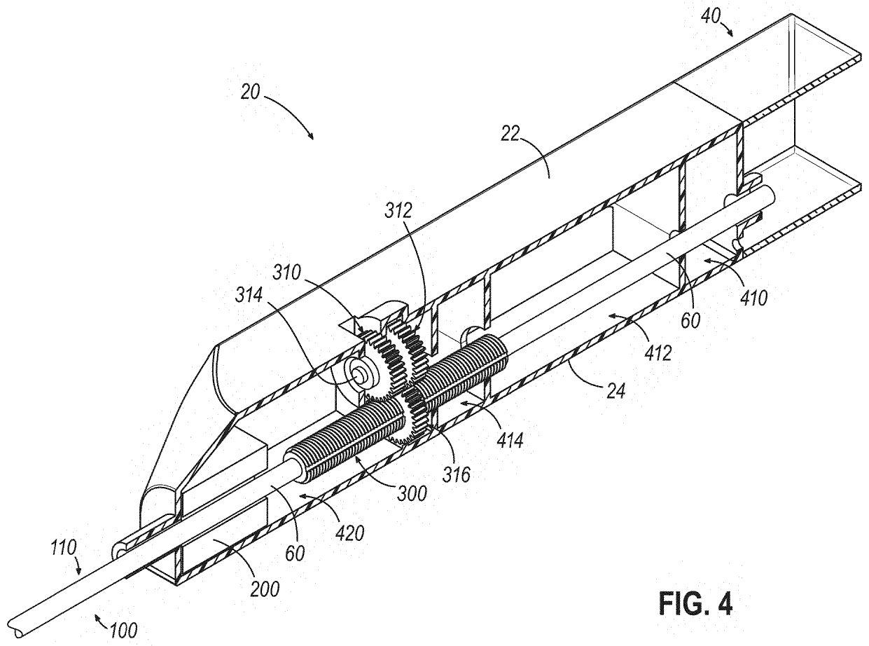

FIG. 4 depicts a cutaway view of probe (20) showing needle assembly (100), a cutter actuation assembly (300), a probe housing (22, 24) and tissue sample holder (40). Needle assembly (100) comprises a needle portion (110) and a valve assembly (200). As will be described in greater detail below, needle assembly (100) is generally operable to pierce tissue where cutter (60) can be positioned to sever a tissue sample from a patient and transport the tissue sample to tissue sample holder (40). More specifically, the needle portion (110) of needle assembly (100) is inserted into a patient's tissue. Cutter actuation assembly (300) is then operable to selectively actuate cutter (60) to an open position after pressing one or more of buttons (54). Once cutter (60) is actuated by cutter actuation assembly (300) into an open position, tissue may be prolapsed into needle portion (110) by means of a vacuum communicated through cutter (60). Cutter (60) may then be selectively actuated by means of cutter actuation assembly (300) into the closed position, severing the prolapsed tissue from the patient. Vent assembly (300) is then operable to selectively vent a portion of needle portion (110) to atmosphere thus creating a pressure differential between proximal and distal ends of the prolapsed tissue. The pressure differential then transports the prolapsed tissue through cutter (60) to tissue sample holder (40).

### A. Exemplary Cutter Actuation Assembly

Cutter actuation assembly (300) comprises a series of gears (310, 312). Gears (310, 312) are configured to simultaneously translate and rotate cutter (60). In the configuration shown, gear (310) is coupled with motor (70) when probe (20) is attached to holster (30) by way of gear (30). In particular, both gears (310, 312) are mounted on a single shaft (314) such that gears (310, 312) rotate together. Thus, gear (310) is driven by gear (34) of holster (30), which also drives gear (312). Gear (312) meshes with a cutter gear (316). As will be described in greater detail below, cutter gear (316) can then drive simultaneous translation and rotation of cutter (60) upon being rotated by gear (310) via gear (312).

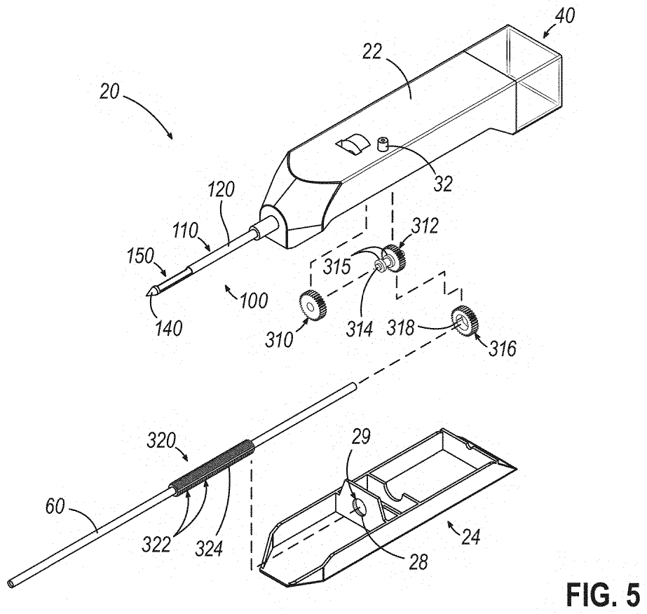

As best seen in FIG. 5, cutter actuation assembly (300) further includes a screw (320) overmolded or otherwise secured to cutter (60) such that screw (320) and cutter (60) rotate and translate unitarily. Screw (320) includes external threading (322) and one or more channels (324) extending through threading (322). One or more channels (324) are configured to slidably engage corresponding protrusions (318) defined by cutter gear (316). In this configuration, rotation of cutter gear (316) is transferred to screw (320), which ultimately transfers rotation to cutter (60).

Threading (322) is configured to engage corresponding internal threading (28) defined within an opening (29) of probe housing (24). As screw (320) is rotated, engagement between threading (322) and threading (28) causes screw (320) to translate relative to probe housing (24). Thus, rotation of cutter gear (316) is generally configured to provide translation of screw (320) and cutter (60) via engagement between threading (322) and threading (28). It should be understood that other configurations may be provided utilizing different gear (310, 312, 316) arrangements. Moreover, configurations involving additional motors (70) may be used. Various suitable motor (70) and gear (310, 312, 316) combinations will be apparent to one of ordinary skill in the art in view of the teachings herein. Indeed, cutter actuation assembly (300) may be constructed in accordance with at least some of the teachings of U.S. Pat. No. 8,206,316, entitled “Tetherless Biopsy Device with Reusable Portion,” issued Jun. 26, 2012, the disclosure of which is incorporated by reference herein. In other examples, cutter actuation assembly (300) may be constructed in accordance with at least some of the teachings of U.S. Pub. No. 2019/0008493, entitled “Apparatus to Allow Biopsy Sample Visualization During Tissue Removal,” published Jan. 10, 2019, the disclosure of which is incorporated by reference herein.

It should be understood that gears (310, 312) are generally fluidly isolated from each other by a seal (315) disposed on shaft (314). In particular, gear (310) is generally exposed to atmosphere such that gear (310) can mesh with gear (34) of holster (30). Meanwhile, gear (312) is fluidly isolated relative to atmosphere. As will be described in greater detail below, this configuration is generally configured to permit vacuum to flow within at least some of the space occupied by gear (312) while still allowing gear (312) to be rotated by gear (34) of holster (30) via gear (310).

### B. Exemplary Needle Portion

FIG. 5 shows an exemplary needle portion (110). Needle portion (110) comprises a cannula (120), a tissue piercing tip (140), and a lateral aperture (150). As is shown, cannula (120) is positioned on top of cannula (120). Although not shown, it should be understood that cannula (120) defines a lumen therein for receiving cutter (60). In some examples, cannula (120) defines multiple lumens therein such as one for receiving cutter (60) and one for transmitting atmospheric air and/or vacuum to lateral aperture (150). Although needle portion (110) of the present example is shown as having a generally round cross-section, it should be understood that other cross-sectional shapes may be used. Indeed, in some examples needle portion (110) can be comprised of a combination of round and oval-shaped tubes to form an oval-shaped cross-section. In other examples, needle portion (110) can be comprised of only circular tubes thus creating a generally figure eight cross-section. Alternatively, needle portion (110) may be comprised of two square tubes thus creating a generally square cross-section. Yet in other configurations, needle portion (110) can be constructed in accordance with at least some of the teachings of U.S. Pat. No. 8,801,742, entitled “Needle Assembly and Blade Assembly for Biopsy Device,” issued Aug. 8, 2014, the disclosure of which is incorporated by reference herein.

Cannula (120) is generally configured to receive cutter (60) and to permit cutter (60) to translate and rotate within a lumen defined by cannula (120). Cannula (120) further comprises lateral aperture (150). Lateral aperture (150) is sized to receive prolapsed tissue during operation of biopsy device (10). Thus, tissue can be received by lateral aperture (150) for severing of a tissue sample by cutter (60) under the influence of vacuum from vacuum pump (50).

In use, cutter (60) can be moved through a variety of positions such as a closed position, an open position and finally in an intermediate position. Each position may correspond to a particular stage in the tissue sample extraction process. For example, the cannula (120) may penetrate a patient's tissue when cutter (60) is in a closed position. In the closed position, cutter (60) is in its furthest distal position relative to lateral aperture (150). Thus, cannula (120) may penetrate through tissue smoothly without catching any surrounding tissue that might impede penetration. In the open position, cutter (60) is in its furthest proximal position relative to lateral aperture (150). This state may, for example, correspond to a position where cannula (120) is oriented inside a patient where a tissue sample may be taken. With cutter (60) in its furthest proximal position relative to lateral aperture (150) a vacuum may be applied to prolapse patient's tissue through lateral aperture (150). Finally, when cutter (60) is in the intermediate position, cutter (60) is in a position between its furthest distal and proximal positions relative to lateral aperture (150). In this position, cutter (60) may be in a motive state from either a closed position or an open position to a closed or open position, respectively. For example, cutter (60) can move from an open to closed position so that cutter (60) may sever a tissue sample. Alternatively, cutter can move from a closed to open position in order to allow the patient's tissue to prolapse through lateral aperture (150). As will be described in further detail below, these various positions correspond to various pneumatic states of valve assembly (200). It should be understood that the various positions of cutter (60) and the corresponding stages in the tissue extraction process are merely exemplary and other suitable combinations will be apparent to one of ordinary skill in the art from the teachings herein.

Tissue piercing tip (140) is shown as having a generally conical body. The shape of tissue piercing tip (140) is merely exemplary and many other suitable shapes may be used. For example, tissue piercing tip (140) may be in the shape of a blade protruding from needle portion (110), disregarding the conical body. Still in further variations, the tissue piercing tip (140) may have a flat blade portion of varying shapes and configurations. Other various configurations for tissue piercing tip (140) and for needle portion (110) in general may be provided as will be apparent to one of ordinary skill in the art in view of the teachings herein. By way of example only, needle portion (110) may be constructed in accordance with at least some of the teachings of U.S. Pat. No. 8,801,742, entitled “Needle Assembly and Blade Assembly for Biopsy Device,” issued Aug. 8, 2014, the disclosure of which is incorporated by reference herein.

### C. Exemplary Valve Assembly

Returning to FIG. 4, probe (20) is shown as including a valve assembly (200).

Valve assembly (200) in the present example is generally configured to provide atmospheric ventilation to needle portion (110). In some examples, this atmospheric ventilation can be supplied between the exterior of cutter (60) and the interior of cannula (120). In other examples, cannula (120) can define a discrete lumen for providing atmospheric air to the distal end of cutter (60). Of course, various alternative configurations can be used as will be apparent to those of ordinary skill in the art in view of the teachings herein.

In the present example, valve assembly (200) is shown schematically. This, it should be understood that valve assembly (200) can take on a variety of forms. For instance, in some examples valve assembly (200) can include a manifold (not shown) and a spool body (not shown). In such examples, the manifold can couple valve assembly (200) to the proximal end of needle portion (110) of needle assembly (100). Meanwhile, the spool body can move relative to one or more vent openings in manifold under the influence of cutter (60) to transition valve assembly (200) from a venting state to a sealed state. By way of example only, the manifold and/or the spool body can be constructed in accordance with at least some of the teachings of U.S. Pat. No. 10,206,665, entitled “Biopsy Device with Translating Valve Assembly,” issued Feb. 19, 2019, the disclosure of which is incorporated by reference herein.

In use, movement of the spool body can be at least partially controlled by movement of cutter (60). For instance, in some examples, valve assembly (200) can be configured to vent the space between cutter (60) and cannula (120) when cutter (60) is disposed in a distal position. Atmospheric air can then freely flow through the manifold and into the space between cutter (60) and cannula (120). Such a position can correspond to severing of a tissue sample using cutter (60). Thus, it should be understood that venting is provided to needle portion (110) after a tissue sample has been severed to promote tissue transport through cutter (60). In other examples, it may be desirable to substantially seal needle portion (110) relative to atmosphere. For instance, in the intermediate position described above, tissue may be prolapsed into lateral aperture (150). In this circumstance, it may be desirable to seal needle portion (110) to prevent escape of vacuum through the interface between cutter (60) can cannula (120). Accordingly, in this circumstance, the spool body can be positioned by cutter (60) such that the spool body seals the manifold. Of course, various other additional or alternative pneumatic states can be used as will be apparent to those of ordinary skill in the art in view of the teachings herein. By way of example only, suitable pneumatic states can be in accordance with at least some of the teachings of U.S. Pat. No. 10,206,665, entitled “Biopsy Device with Translating Valve Assembly,” issued on Feb. 19, 2019, the disclosure of which is incorporated by reference herein.

### D. Exemplary Integrated Vacuum Reservoir

In some examples it may be desirable to provide a biopsy device such as biopsy device (10) with one or more reservoirs for vacuum. For instance, some tethered biopsy devices may generally use one or more vacuum canisters in an external vacuum system. Use of vacuum canisters can be desirable to provide additional volume for vacuum. This additional volume can make the vacuum system overall more resistant to reduced vacuum pressure caused by sudden fluctuations in vacuum flow during a biopsy procedure. In other words, additional volume for vacuum can make vacuum pressure more consistent over time. Increased consistency in vacuum pressure is generally desirable to permit larger samples sizes, increased response time, and improved transport of samples through the biopsy device.

By contrast, in tetherless biopsy devices such as biopsy device (10), vacuum canisters may generally not be used due to the entirety of the vacuum system being integrated into the biopsy device itself. Without vacuum a vacuum canister or other similar structure, the overall volume of the vacuum system is reduced. This reduction in volume may result in the vacuum system being more susceptible to sudden fluctuations in vacuum flow resulting in more erratic vacuum pressures over the course of a biopsy procedure. Thus, in some circumstances it may be desirable to incorporate structures and features into a biopsy device to provide additional volume to a vacuum system. Although various exemplary biopsy device configurations are described below, it should be understood that various modifications can be made without departing from the spirit of the examples disclosed herein.

As best seen in FIG. 5, probe (20) is formed of an upper housing (22) and a lower housing (24). Both upper housing (22) and lower housing (24) are configured to couple to each other to form a fluid tight seal such that the interior of probe (20) is generally sealed relative to atmosphere. To facilitate sealing, lower housing (24) defines a geometric shape that is generally free from hard corners and/or edges. Additionally, the proximal and distal end of lower housing (24) is tapered to provide self-centering when lower housing (24) is coupled to upper housing (22). This configuration is generally configured to permit lower housing (24) to readily seal with upper housing (22) to thereby seal the interior of probe (20) relative to atmosphere. Although lower housing (24) (and corresponding portions of upper housing (22)) of the present example is shown as having a specific geometric shape, it should be understood that various alternative geometric shapes can be used provided that such shapes are generally free of hard edges and corners, and/or self-centering.

Upper housing (22) and lower housing (24) of the present example are generally configured to be coupled by way of ultrasonic welding. Thus, the specific geometric shape of lower housing (24) is configured to promote adherence to upper housing (22) during ultrasonic welding through the absence of hard edges and corners and the self-centering configuration of upper housing (22) and lower housing (24). Although use of ultrasonic welding is described herein as being suitable to couple upper housing (22) and lower housing (24), it should be understood that in other examples various alternative coupling mechanisms can be used. For instance, in some examples upper housing (22) and lower housing (24) can be coupled by an adhesive such as epoxy. In other examples, upper housing (22) and lower housing (24) can be coupled by mechanical fastening with one or more gaskets disposed between upper housing (22) and lower housing (24) to provide sealing. Still other examples of coupling for upper housing (22) and lower housing (24) can be used as will be apparent to those of ordinary skill in the art in view of the teachings here.

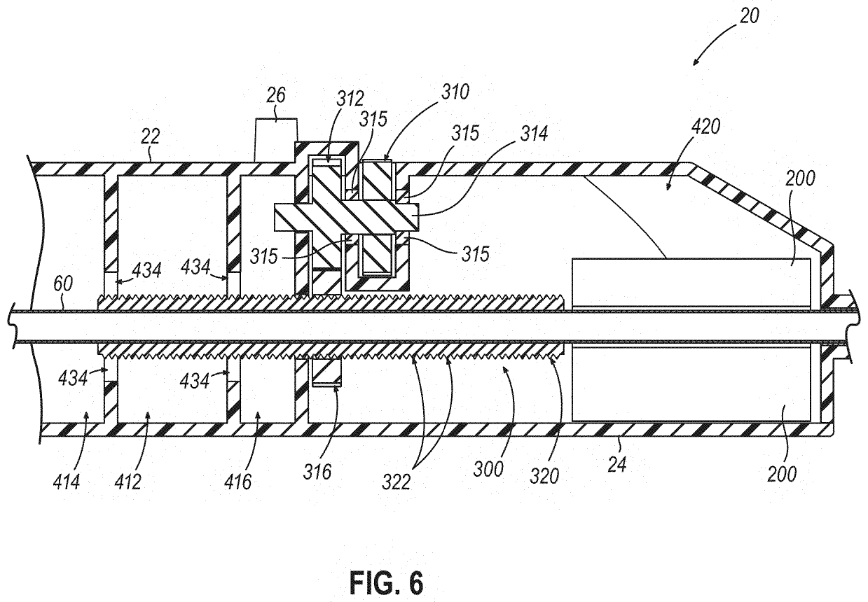

FIGS. 4, 6, and 7 show detailed view of the interior of probe (20). As can be seen, upper and lower housings (22, 24) together define one or more vacuum reservoirs or chambers (410, 412, 414, 416) and a vent chamber (420) therein. Vacuum reservoirs (410, 412, 414, 416) are all generally configured to communicate fluid between each other to provide flow of vacuum from port (24) to tissue sample holder (40). As will be described in greater detail below, vacuum reservoirs (410, 412, 414, 416) are generally configured to provide increased volume to the vacuum system to make the vacuum system overall more resistant to sudden changes in vacuum flow during a procedure such as a biopsy procedure.

The shape of each vacuum reservoir (410, 412, 414, 416) is generally defined by the construction of upper and lower housings (22, 24). For instance, in the present example upper and lower housings (22, 24) define one or more internal walls (430) that define separate compartments within the interior of probe (20) that correspond to each vacuum reservoir (410, 412, 414, 416). In the present example, three internal walls (430) are used to form four separate vacuum reservoirs (410, 412, 414, 416). However, it should be understood that in other examples different wall configurations can be used to provide different corresponding vacuum reservoir (410, 412, 414, 416) configurations. Indeed, in the present example the particular construction of internal walls (430) is merely to provide stiffening to probe (20). Thus, in other examples more or less internal walls (430) could be used or even eliminated entirely depending on the desired physical properties of probe (20). Moreover, although internal walls (430) of the present example are shown as dividing the interior of probe (20) vertically, in other examples, internal walls (430) can divide the interior of probe (20) horizontally, or a combination of vertically and horizontally.

Internal walls (430) generally include one or more openings (432) to promote fluid flow between each vacuum reservoir (410, 412, 414, 416). Openings (432, 434) can take on a variety of forms. For instance, in the present example some internal walls (430) include fluid openings (432) configured to merely accommodate the flow of fluid. Meanwhile, other internal walls (430) include cutter openings (434) configured to accommodate movement of cutter (60), one or more components of cutter actuation assembly (300), and the flow of fluid. In either style of opening (432, 434), it should be understood that each opening (432, 434) is generally configured to not impede the flow of vacuum during a procedure. Thus, the flow rate of vacuum through a given opening (432, 434) is generally greater than the flow rate of the vacuum system overall so as to not impede operation of the vacuum system. Alternatively, each internal wall (430) can include multiple openings (432, 434) of various configurations to likewise promote the flow of vacuum without impeding operation of the vacuum system.

Upper probe housing (22) and lower probe housing (24) further define a vent chamber (420) disposed distally of vacuum reservoirs (410, 412, 414, 416). Vent chamber (420) is generally fluidly isolated from vacuum reservoirs by distal wall (422). Thus, it should be understood that distal wall (422) is configured to isolate vent chamber (420) from vacuum reservoirs (410, 412, 414, 416). As such, distal wall (422) can include various seals, gaskets, or other features to permit various operational components such as cutter actuation assembly (300) to pass through distal wall (422) while maintaining fluid isolation.

Vent chamber (420) is generally configured to provide a space for valve assembly (200) to operate. As described above, valve assembly (200) can be configured to provide ventilation to atmosphere to needle assembly (100). Thus, in some examples upper probe housing (22) and/or lower probe housing (24) can include various external vents or vent passages to maintain vent chamber (420) at atmospheric pressure. However, it should be understood that vent chamber (420) being at atmospheric pressure is not required. For instance, in some examples valve assembly (200) itself can be fluidly isolated from the rest of probe (20) and have a direct fluid connection to atmosphere through a tube or passageway. In such examples, vent chamber (420) can be used as another vacuum reservoir similar to vacuum reservoirs (410, 412, 414, 416) described above. Thus, it should be understood that in some examples distal wall (422) can likewise include openings similar to openings (432, 434) described above to promote use of vent chamber (420) as another vacuum reservoir.

As described above, gears (310, 312) are generally fluidly isolated from each other by a seal (315) disposed on shaft (314). This fluid isolation is generally configured to promote fluid isolation of vacuum reservoirs (410, 412, 414, 416) relative to the exterior of probe (20). For instance, gear (310) is generally exposed to the exterior of probe (20) to mesh with gear (34) of holster (30). Meanwhile, gear (312) is disposed within the interior of upper probe housing (22) and lower probe housing (24) and is thus fluidly isolated from the exterior of probe (20). Seal (315) provides fluid isolation between gears (310, 312) by sealingly engaging shaft (314). Thus, gear (310) is configured to drive rotation of gear (312) at atmospheric pressure, while gear (312) is exposed to some vacuum pressure without creating any fluid leak passages between gears (310, 312). Accordingly, gears (310, 312) can together provide motive power to cutter actuation assembly (300) from holster (30) without substantially disrupting operation of vacuum reservoirs (410, 412, 414, 416).

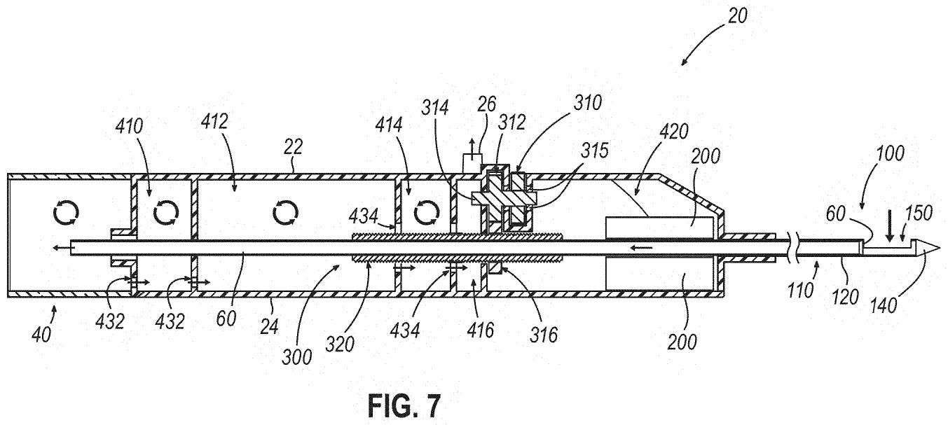

FIG. 7 provides an exemplary view of the flow of vacuum through probe (20). As can be seen, vacuum is provided by holster (30) at port (26). Vacuum is then pulled through port (26) and into vacuum reservoir (416) where vacuum can freely circulate within vacuum reservoir (416). Vacuum can then pass freely through opening (434) in internal wall (430) adjacent to vacuum reservoir (416) into vacuum reservoir (414). Vacuum can likewise freely circulate within vacuum reservoir (414). Vacuum can then freely pass through opening (434) in internal wall (430) between vacuum reservoir (414) and vacuum reservoir (412) into vacuum reservoir (412). Vacuum can then freely circulate within vacuum reservoir (412). Vacuum can then freely pass through opening (432) in internal wall (430) between vacuum reservoir (412) and vacuum reservoir (410) into vacuum reservoir (410). Vacuum can then freely circulate within vacuum reservoir (412).

Once vacuum has passed through all vacuum reservoirs (410, 412, 414, 416), vacuum can pass through opening (432) in internal wall (430) adjacent to tissue sample holder (40) and into tissue sample holder (40). From tissue sample holder (40), vacuum can enter cutter (60) where it can travel through cutter (60) to draw a tissue sample through lateral aperture (150) during a sampling sequence. Cutter (60) can then sever the tissue sample and the vacuum can be used to transport the severed tissue sample through cutter (60) and into tissue sample holder (40). This sampling sequence can then be repeated as desired to collect multiple tissue samples within tissue sample holder (40).

It should be understood that the sampling sequence described above can result in variable vacuum flow at various stages of the sampling sequence. For instance, vacuum flow may be relatively high during suction of a tissue sample through lateral aperture (150). Vacuum flow may also be relatively high during transport of a severed tissue sample through cutter (60). Under other circumstances, vacuum flow may be relatively low at other stages such as during severing of a tissue sample. In still other circumstances, vacuum flow may be relatively moderate or nominal such as during retraction of cutter (60) relative to lateral aperture (150). Thus, it should be understood that periods of relatively high vacuum flow can result in relatively high volumetric consumption of vacuum while period of relative low vacuum flow can result in relatively low volumetric consumption of vacuum. Nonetheless, during all these stages, holster (30) provides a continuous flow of vacuum regardless of the particular stage of the sampling sequence. As a consequence, cutter (60) and tissue sample holder (40) can draw on vacuum reservoirs (410, 412, 414, 416) to consume more or less volumetric vacuum without overwhelming the continuous flow of vacuum provided by holster (30). Overall, this results in a more continuous vacuum pressure over time.

The particular amount of volume provided by vacuum reservoirs (410, 412, 414, 416) can vary depending on the particular configuration of vacuum reservoirs (410, 412, 414, 416). Although vacuum reservoirs (410, 412, 414, 416) can be configured in a variety of ways to provide a variety of specific volumes, the present example is configured to provide approximately 10 to 20 times more volume relative to a vacuum system connected directly to tissue sample holder (40). In some examples, vacuum reservoirs (410, 412, 414, 416) collectively provide a volume that is 10 to 20 times greater than the volume of tissue sample holder (40). In other examples, vacuum reservoirs (410, 412, 414, 416) collectively provide a volume that is approximately 175 cc. Of course, various other alternative volumes can be used as will be apparent to those of ordinary skill in the art in view of the teachings herein.

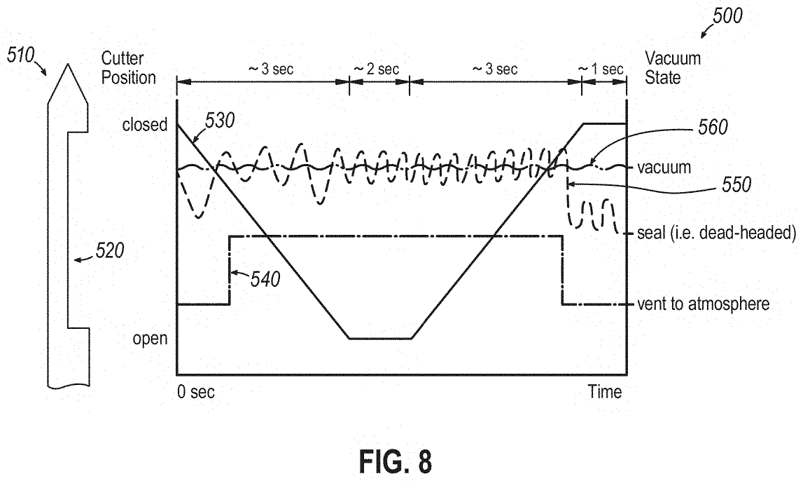

FIG. 8 depicts the principles described above schematically. In particular, FIG. 8 shows an algorithm (500) including movements of cutter (60) in relation to cannula (120), which is represented by graphical representation (510) including a graphical representation (520) of lateral aperture (150). Movement of cutter (60) is shown in line (530) for a full range of travel for cutter (60). Line (540) represents pneumatic states of valve assembly (200) during the tissue sampling sequence.

The pneumatic state of the lumen extending through cutter (60) is shown by line (550) and line (560). Here, separate lines are provided to provide a comparison between the present example and an example without vacuum reservoirs (410, 412, 414, 416) (e.g., vacuum supplied directly to tissue sample holder (40)). For instance, line (550) shows an example of vacuum pressure over time in an example without vacuum reservoirs (410, 412, 414, 416).

As can be seen, vacuum pressure shown by line (550) varies substantially over time. These changes in vacuum pressure result from changes to volumetric consumption of vacuum increasing throughout the sampling sequence thereby leading to overloading of the continuous vacuum pressure supplied by holster (30). By contrast, line (560) shows vacuum pressure over time in the present example that includes vacuum reservoirs (410, 412, 414, 416). As can be seen, vacuum pressure is substantially stabilized over time due to additional volume for expansion and contraction of vacuum provided by vacuum reservoirs (410, 412, 414, 416). Thus, it should be understood that vacuum reservoirs (410, 412, 414, 416) in the present example are configured to generally provide a vacuum pressure smoothing effect over time. This effect is generally desirable for increased response time, improved transport of tissue samples, collection of larger tissue samples, and more consistent and reliable operation of biopsy device (10). Although lines (550, 560) show certain specific frequencies and amplitude of vacuum pressure over time, it should be understood that in other examples the frequency and amplitude of vacuum pressure over time may vary depending on a variety of conditions. Indeed, it should be understood that lines (550, 560) are primarily to show the conceptual differences described above.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

### V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A probe for use with a biopsy device having a tissue sample holder defining a sample chamber and a holster removably secured to the probe, the probe comprising a housing defining a vacuum chamber within the housing, wherein the vacuum chamber is in communication with the sample chamber of the tissue sample holder.

### EXAMPLE 2

The probe of Example 1, further comprising a cutter for severing a tissue sample, wherein the cutter extends through the vacuum chamber and is in communication with the tissue sample holder.

### EXAMPLE 3

The probe of Example 1, further comprising a cutter drive and a cutter for severing a tissue sample, wherein the cutter drive is configured to translate and rotate the cutter, wherein the cutter drive and the cutter are both partially disposed within the vacuum chamber.

### EXAMPLE 4

The probe of any one or more of Examples 1 through 3, wherein the vacuum chamber includes four fluid reservoirs separated by a plurality of internal walls.

### EXAMPLE 5

The probe of any one or more of Examples 1 through 3, wherein the vacuum chamber includes a plurality of fluid reservoirs separated by one or more internal walls extending within the probe axially, laterally, or a combination thereof

### EXAMPLE 6

The probe of any one or more of Examples 1 through 5, wherein the vacuum chamber defines a first volume, wherein the first volume is greater than a second volume defined by the tissue sample holder.

### EXAMPLE 7

The probe of any one or more of Examples 1 through 5, wherein the vacuum chamber defines a first volume, wherein the first volume is 10 to 20 times greater than a second volume defined by the tissue sample holder.

### EXAMPLE 8

The probe of any one or more of Examples 1 through 7, wherein the housing of the probe includes an upper housing and a lower housing coupled together and defining an interface, wherein the interface is substantially free of sharp edges.

### EXAMPLE 9

The probe of any one or more of Examples 1 through 7, wherein the housing of the probe includes an upper housing and a lower housing coupled together and defining an interface, wherein the lower housing is self-centering relative to the upper housing.

### EXAMPLE 10

The probe of any one or more of Examples 1 through 9, further comprising a first gear and a second gear rotatably connected by a shaft, wherein the first gear is exposed relative to the exterior of the housing, wherein the second gear is disposed within the vacuum chamber and is sealed relative to the exterior of the housing by a seal engaged with the shaft.

### EXAMPLE 11

A handheld tetherless biopsy device for collecting one or more tissue samples, wherein the biopsy device comprises: a holster having a motor and a vacuum pump coupled to the motor; a probe removably coupled to the holster, wherein the probe includes a housing having a vacuum port in communication with the vacuum pump of the holster, wherein the housing defines a vacuum reservoir in communication with the vacuum port; and a tissue sample holder defining a sample chamber configured to receive tissue samples therein, wherein the sample chamber is in communication with the vacuum reservoir of the probe.

### EXAMPLE 12

The biopsy device of Example 11, wherein the vacuum reservoir includes a plurality of reservoirs separated by one or more internal walls and interconnected by an opening disposed within each of the one or more internal walls.

### EXAMPLE 13

The biopsy device of Examples 11 or 12, wherein the probe further includes a cutter and a cutter drive configured to translate and rotate the cutter, wherein at least a portion of the cutter and the cutter drive is disposed within the vacuum reservoir.

### EXAMPLE 14

The biopsy device of Example 11 or 12, wherein the probe further includes a cutter and a cutter drive configured to translate and rotate the cutter, wherein at least a portion of the cutter and the cutter drive is disposed within the vacuum reservoir, wherein the opening of at least one of the one or more internal walls is configured to receive the cutter and a portion of the cutter drive while providing fluid flow between two fluid reservoirs of the plurality of fluid reservoirs.

### EXAMPLE 15

The biopsy device of any one or more of Examples 11 through 13, wherein the probe further includes a valve assembly configured to provide selective ventilation to a portion of the probe, wherein the valve assembly is fluidly isolated from the vacuum reservoir.

### EXAMPLE 16

The biopsy device of any one or more of Examples 11 through 13, wherein the probe further includes a vent chamber and a valve assembly disposed within the vent chamber and configured to provide selective ventilation to a portion of the probe, wherein the vent chamber is fluidly isolated from the vacuum reservoir.

### EXAMPLE 17

The biopsy device of any one or more of Examples 11 through 13, wherein the probe further includes a valve assembly configured to provide selective ventilation to a portion of the probe, wherein the valve assembly is fluidly isolated from the vacuum reservoir while also being disposed within a portion of the vacuum reservoir.

### EXAMPLE 18

The biopsy device of any one or more of Examples 11 through 17, wherein the vacuum reservoir defines a total fluid volume of about 175 cc.

### EXAMPLE 19

The biopsy device of any one or more of Examples 11 through 17, wherein the housing of the probe includes a first housing and a second housing, wherein the first housing and the second housing are configured to couple together to seal the vacuum reservoir relative to the exterior of the probe.

### EXAMPLE 20

The biopsy device of any one or more of Examples 11 through 19, wherein the vacuum pump is continuously driven by the motor.

### EXAMPLE 21

A method for use with a biopsy device having a probe, a tissue sample holder, and a holster, the method comprising: inserting a needle of the probe into tissue; translating a cutter within the needle distally to sever a tissue sample; drawing a vacuum pressure supplied by a vacuum pump within the holster from a vacuum reservoir defined by a housing of the probe to transport the severed tissue sample through the cutter and into the tissue sample holder.

### EXAMPLE 22

The method of Example 21, wherein the step of drawing the vacuum pressure includes supplying a continuous flow of vacuum from the vacuum pump of the holster to the vacuum reservoir.

### EXAMPLE 23

The method of Example 22, wherein the vacuum pressure during the step of drawing the vacuum pressure is substantially continuous as the severed tissue sample is transported through the cutter.

### EXAMPLE 24

The method of any one or more of Examples 21 through 23, further comprising translating the cutter within the needle proximally after transporting the severed tissue sample through the cutter; and drawing another vacuum pressure supplied by the vacuum pump within the holster from the vacuum reservoir while the cutter is translated proximally.

### EXAMPLE 25

The method of Example 24, wherein the other vacuum pressure is substantially the same as the vacuum pressure suppled during transport of the severed tissue sample.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A probe for use with a biopsy device, the biopsy device having a tissue sample holder defining a sample chamber and a holster removably secured to the probe, the probe comprising:
a housing defining a vacuum chamber within the housing, the vacuum chamber being in communication with the sample chamber of the tissue sample holder.

2. The probe of claim 1, further comprising a cutter for severing a tissue sample, the cutter extending through the vacuum chamber and is in communication with the tissue sample holder.

3. The probe of claim 1, further comprising a cutter drive and a cutter for severing a tissue sample, the cutter drive being configured to translate and rotate the cutter, the cutter drive and the cutter being both partially disposed within the vacuum chamber.

4. The probe of claim 1, the vacuum chamber including four fluid reservoirs separated by a plurality of internal walls.

5. The probe of claim 1, the vacuum chamber including a plurality of fluid reservoirs separated by one or more internal walls extending within the probe axially, laterally, or a combination thereof

6. The probe of claim 1, the vacuum chamber defining a first volume being greater than a second volume defined by the tissue sample holder.

7. The probe of claim 1, the vacuum chamber defining a first volume, the first volume being 10 to 20 times greater than a second volume defined by the tissue sample holder.

8. The probe of claim 1, the housing of the probe including an upper housing and a lower housing coupled together and defining an interface, the interface being substantially free of sharp edges.

9. The probe of claim 1, the housing of the probe including an upper housing and a lower housing coupled together and defining an interface, the lower housing being self-centering relative to the upper housing.

10. The probe of claim 1, further comprising a first gear and a second gear rotatably connected by a shaft, the first gear being exposed relative to the exterior of the housing, the second gear being disposed within the vacuum chamber and is sealed relative to the exterior of the housing by a seal engaged with the shaft.

11. A handheld tetherless biopsy device for collecting one or more tissue samples, the biopsy device comprising:
a holster having a motor and a vacuum pump coupled to the motor;
a probe removably coupled to the holster, the probe including a housing having a vacuum port in communication with the vacuum pump of the holster, the housing defining a vacuum reservoir in communication with the vacuum port; and
a tissue sample holder defining a sample chamber configured to receive tissue samples therein, the sample chamber being in communication with the vacuum reservoir of the probe.

12. The biopsy device of claim 11, the vacuum reservoir including a plurality of reservoirs separated by one or more internal walls and interconnected by an opening disposed within each of the one or more internal walls.

13. The biopsy device of claim 11, the probe further including a cutter and a cutter drive configured to translate and rotate the cutter, at least a portion of the cutter and the cutter drive being disposed within the vacuum reservoir.

14. The biopsy device of claim 11, the probe further including a cutter and a cutter drive, the cutter drive being configured to translate and rotate the cutter, at least a portion of the cutter and the cutter drive being disposed within the vacuum reservoir, the opening of at least one of the one or more internal walls being configured to receive the cutter and a portion of the cutter drive while providing fluid flow between two fluid reservoirs of the plurality of fluid reservoirs.

15. The biopsy device of claim 11, the probe further including a valve assembly configured to provide selective ventilation to a portion of the probe, the valve assembly being fluidly isolated from the vacuum reservoir.

16. The biopsy device of claim 11, the probe further including a vent chamber and a valve assembly disposed within the vent chamber and configured to provide selective ventilation to a portion of the probe, the vent chamber being fluidly isolated from the vacuum reservoir.

17. The biopsy device of claim 11, the probe further including a valve assembly configured to provide selective ventilation to a portion of the probe, the valve assembly being fluidly isolated from the vacuum reservoir while also being disposed within a portion of the vacuum reservoir.

18. The biopsy device of claim 11, the vacuum reservoir defining a total fluid volume of about 175 cc.

19. The biopsy device of claim 11, the housing of the probe including a first housing and a second housing, the first housing and the second housing being configured to couple together to seal the vacuum reservoir relative to the exterior of the probe.

20. A method for use with a biopsy device having a probe, a tissue sample holder, and a holster, the method comprising:
inserting a needle of the probe into tissue;
translating a cutter within the needle distally to sever a tissue sample; and
drawing a vacuum pressure supplied by a vacuum pump within the holster from a vacuum reservoir defined by a housing of the probe to transport the severed tissue sample through the cutter and into the tissue sample holder.
